(19) United States
(12) Patent Application Publication
(10) Pub. No.: US 2008/0021267 A1
(43) Pub. Date: Jan. 24, 2008

(54) Glans-Stripper Apparatus To Hold The Tucked Up Foreskin At The Root Of The Erect Penis

(76) Inventor: Maarten Willem Driessin, Amsterdam (NL)

Correspondence Address: BROOKS KUSHMAN P.C., 1000 TOWN CENTER, TWENTY-SECOND FLOOR, SOUTHFIELD, MI, 48075, US

(21) Appl. No.: 10/599,642

(22) PCT Filed: Apr. 4, 2005
(86) PCT No.: PCT/NL05/00249

§ 371 Date: Oct. 4, 2006

(30) Foreign Application Priority Data
Apr. 4, 2004 (NL) 1025879
Nov. 1, 2004 (NL) 1027388

(51) Int. Cl. A61F 5/00

(52) U.S. Cl. 600/41

(57) ABSTRACT

The Glans-stripper provides a naked glans during sex by holding the tucked up foreskin at the root of the erection leaving the hands free for other use. The more sensitive inside layer of the foreskin will be stretched outwardly all over the erection and the completely naked glans will be stretched out by pulling its skin tight with its bridle. If wanted the Glans-stripper can easily be constructed by the consumer himself using the loose parts. A do-it yourself kit can he made out of a combination of these loose parts. Loose parts like a spherical shaped and beadlike form K(-) made out of two hinging parts with a clasp to fasten them up. A length of about 15 meters of flat cotton (sports) shoelace material rolled up around a piece of carton. Elastic ribbon made out of two pieces of elastic ribbon connected to each other by a thin but very strong thread making thin but very strong separations between interruptions of a buttonhole long. Small tube like forms with a cut from one end to the other that can be glued around the ends of the shoe-lace material to prevent them from raveling.

The Glans-stripper is meant for men with a glans that is covered by their foreskin during an erection. The Glans-stripper provides a naked glans during sex by holding the tucked up foreskin at the root of the penis leaving the hands free for other things. The more sensitive inside layer of the foreskin will be turned to the outside to be stretched all over the erection and the sensitive glans will be totally naked and stretched out because of its skin being pulled tight by its bridle that is being pulled tight by the tucked up fore-skin.

All of the designs of the Glans-stripper and the parts described in this application ought to be made from material that is of no risk to the health, does not taste bad and is not unpleasant to touch with the teeth.

The Glans-stripper is made of a part A that can hold the tucked up foreskin at the root of the erection and a part B that can hold A by connecting A to rest of the body so A will not slide back together with the foreskin. I have made three basic designs.

In Design 1 part A is a piece of elastic ribbon (which is called in the Netherlands ‘sportelastiek’) whose ends are indissoluble connected to each other and to the middle of part B. The outline of the circle formed by A should be in between ⅔ and % of the outline formed by the root of the erection to be functional for most men. The width of the elastic ribbon should be 1 to 1½ cm. If the outline of A is to big the foreskin will be slipping and if the outline of A is to small the circulation of the blood will be congealed. Part B is made of the same shoe-lace material as a flat cotton shoe-lace that is commonly used in sports shoes. Part A and part B can be sewed together or they can be tied together or they can be riveted together with a rivet. Also glue could be used. Both halves of part B are led through the same spherical shaped and beadlike form K(1) that clenches the shoe-lace material V so tight K(1) will not move during normal use of the Glans-stripper but can be moved if wanted to adjust the size by using force or by performing a particular action. Part A will hold tight the foreskin at the root of the erection and both halves of B will go from the upper side of the root of the erection along both sides of the root of the scrotum in the direction of the anus around where K(1) holds both halves of B together so they can split and go along both sides of the hips to the front of the body to be tied together like shoe-laces. If necessary both halves of B can be connected to each other by using a fastener and the length of both halves could be adjusted like a guy-rope of a tent.

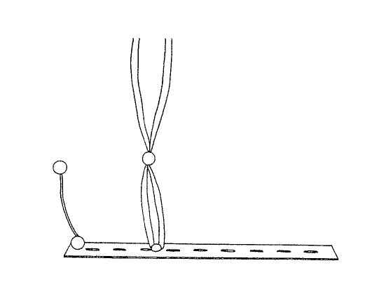

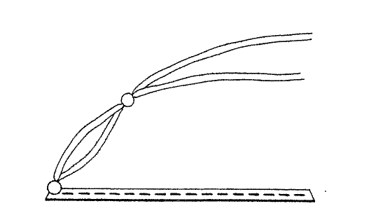

In Design 2 (FIG. 2) A is a piece of elastic ribbon with buttonholes. The buttonholes are in line with each other and the fastener is a spherical shaped and beadlike form K(2). K(2) has connected to it on one side an end of the elastic ribbon and on the other side the middle of part B. Both halves of part B must be led through the buttonhole that is needed to make the desired size of part A. By pulling part B and the other end of the elastic ribbon at the same time Design 2 can be closed easily when it is placed and afterwards it can be opened just as easily as well. A variation to Design 2 (FIG. 1) is not to connect part B at an end of the elastic ribbon to K(2) but more to the middle of the elastic ribbon. The fastener will still be formed by K(2) but in stead of part B a piece of shoe-lace material with on the other end of it an other beadlike form K(3) will be connected to K(2). If K(3) is pushed through the needed buttonhole this variation on Design 2 can easily be closed when it is placed by pulling K(3) and the other end of the elastic ribbon at the same time.

In Design 3 part A is made using part B by leading the middle of part B doublefolded through an extra beadlike form K(4) and a form PK which looks like a flattened version of K(4). This way the middle of part B will form a noose that can be used as part A. PK can be like a little discus with a hole. K(4) and PK need to clench the shoe-lace material V so tight that one has to use force to move them. Both ends of B has to be led through the middle of the two parts of B that will be in between K(4) and PK when they have not been pulled together completely. By holding K(4) and by pulling B at the same time K(4) and PK can be pulled together which results in an extra manner of holding tight B. The total amount of resistance B has to undergo must be big enough to make the size of A a stable one. After leaving K(4) the two halves of B should be led through an extra independent noose L before they are led through the middle of the two parts of B that are in between K(4) and PK. The independent noose L should be big and firm enough to be held firmly. L can be made from a piece of shoe-lace material. By pulling L part B can be pulled back from in between K(4) and PK so there will be enough room to pull K(4) and PK to enlarge the size of A that is enclosing the erection. If part B has to be able to surround the erection too its length should be adjusted by enlarging it. The size of part A can best be determined up front and be placed around the penis before it is erected.

Design 1 too can best be placed around the penis before it is erected. To place it around the erection it can be stretched around the fingers to bring it past the glans. By holding the erection by its skin and by holding part B close to part A part A can be moved along the erection bit by bit by moving it from one side to the other over and over again.

Also a cylindrical form made of firm but flexible plastic just small enough to stretch part A of Design 1 as far as possible and thin enough to be brought to the root of the erection without being to small. The cylindrical form can be like a rolled up rectangle whose endings can be shoven over one another so the consumer can adjust the size himself.

The Glans-stripper can be made out of loose parts and if wanted in a simple manner by the consumer himself.

A part of the Glans-stripper is the elastic ribbon with buttonholes of Design 2. If the seperations between the buttonholes are kept as small as possible the minimal distance required to adjust the size of part A can be kept as small as possible. The minimal distance of the buttonholes depends upon the size of K(2). It should be easy to pull K(2) through the buttonhole that is needed just by pulling part B. The elastic ribbon with the buttonholes can be formed by two pieces of elastic ribbon that are tied together with a thin but very firm thread with interruptions of a buttonhole long. A thread thin but firm enough to keep the seperations thin but firm enough so the middle of part B can be folded around it to construct Design 2 without creating an uncomfortable lump as K(2) is placed as tight as possible to the elastic ribbon. A thread thin but firm enough to make sure the elastic ribbon with the buttonholes will not have to endure the heavy wear and tear that would appear if it was made from the same materials common elastic ribbon with buttonholes is made from. To create a separation the thin but very firm thread can be led through both of the elastic ribbons near an edging and then be knotted together. To avoid a thickening because of the knot the thin but very strong thread can be laced through the material of the elastic ribbons. In the case of customary elastic ribbon with buttonholes the thread that is to weak to make suitable elastic ribbon containing buttonholes with durable and very strong but very small seperations could be replaced by a thread that is strong enough to create suitable seperations that are firm and durable and thin enough. A total width of the elastic ribbons of 1 to 1½ cm could be enough. Also more than two pieces of elastic ribbon could be used being connected to each other in the above manner to create several rows of buttonholes in between them. If in these several rows the starting points of the buttonholes are not on a level with each other the size of part A can be adjusted with a length less than a buttonhole long. The pieces of elastic ribbon can also be connected using an elastic cord which goes from the edging of one ribbon to the edging of the other ribbon and up and fro between them using steps of half a buttonhole long. The elastic ribbon and the elastic cord can be connected to one another using a thin but very strong thread so it can be used to connect Part B to it. On the ribbon the length could be indicated in centimeters and it could be sold for example 3 meters in length and rolled around a piece of carton. As decoration or for pleasure or stimulation a structure could be attached to the elastic ribbon.

A part of the Glans-stripper is the shoe-lace material V that is commonly used for making fiat plaited cotton laces meant for sports shoes. It could be sold for example 15 meters in length and rolled around a piece of carton so the consumer can make several parts B himself. On V the length could be indicated in centimeters.

A part of the Glans-stripper is the spherical shaped and beadlike form K(-) with a hole. If K(-) is one part in whole V could be glued to the inside of the hole. Through an extra hole which leads to the middle of the hole containing V glue could be injected. In stead of using glue the extra hole could contain screw-thread and a screw to press V firmly against the side of its hole. The extra hole goes straight through K(-) containing a pin/bolt that crosses the hole of V only partially. The pin/bolt contains free space on its crossing with the hole of V. By turning the pin/bolt the free space will be replaced by a piece of the pin/bolt resulting in pressing V tight because there is not enough room left for V anymore. If the extra hole has no screw-thread it should enclose the pin/bolt so tight it can be turned but not fall out.

K(-) can also be made out of two parts. One part containing a gully and the other part containing a ledge. The ledge fits the gully and together they can hold V tied in between them. The two parts of K(-) can be hold firmly together using two countersinked screws. The two parts can also be connected through a hinge and be pressed together with a fastener like a screw or a clasp. The fastener and the hinge an the clasp can be countersinked to prevent the consumers from damaging themselves. On the bottom of the gully and on the top of the ledge there can be a raw or rugged structure to have more grip on V.

A part of Design 3 is a small discus like and beadlike form PK. PK is a flattened version of form K(-). PK and K(4) can be integrated with one another to look more like one spherical shaped and beadlike form. Instead of one hole K(-) can contain two holes through which V can be led. If necessary the holes can have a different diameter so the smallest hole will have more grip on V than the other one.

K(-) can be a pointed screw with a head as flat as the head of the lower part of the rivet, the flat head of the lower part of the rivet the little tube is connected to. The pointed screw can be pushed through the elastic ribbon and through V. A little spherical shaped and beadlike form could be turned onto the screw to squeeze it all together. In Design 1 this could be used to connect the elastic ribbon of part A to itself and to the shoe-lace material of part B.

All kinds of shapes or structures could be attached to K(-) or any other part of the Glans-stripper as decoration or to stimulate.

A part C of the Glans-stripper which is like a small rectangle of firm plastic which is bend into a little archer's bow. The endings of the little bow can be bend outwards and they contain a little slit through which V or the elastic ribbon can be led. C can take the pressure of V or the elastic ribbon to spare the tube through which the sperm has to travel outside during an ejaculation.

A part R of a Glans-stripper consisting of a little rolled up rectangle which looks like a little piece of a thin drinking-straw containing a cut from one end to the other. R can be placed around the ends of the shoe-lace material and than be glued to it to prevent it from unraveling. R could be made out of shrinking stocking/plastic with if necessary a raw or rugged structure on the inside to have more grip on the shoe-lace material.

After being heated the piece of shrinking stocking/plastic should shrink to enclose the shoe-lace material tightly.

A do-it-yourself kit could be made out of a combination of the mentioned parts of the Glans-stripper that are needed to make one or several of the Designs. The contents can be supplied with a needle and some strong (fishing) thread, rivettes, a small piercer or (metal) pricker that fits into the hole of the little tube that is a part of the lower part of the rivet, glue and some bags or small boxes the selfmade Glans-strippers can be kept in.

The strong (fishing) thread can be used to pull the shoe-lace material through the elastic ribbon or through the hole of K(-) if K(-) is made out of one part.

Below I will try to give a short impression of why my inventions are an improvement compared to the present state of the technology.

Because part A of Design 1 is made out of elastic ribbon without any fastener enlarging the risk of damaging parts of the body.

Because Design 1 and 2 can hold the foreskin at the root of the erection without hindering the circulation of the blood seriously and without forcing the erection to take on a different shape and it does not take much space.

Because B does not connect A to the scrotum nor does it connect A behind the back to some kind of belt.

Because B is led alongside the root of the scrotum in the direction of the anus around where it is brought together in order to be able to split and go around the body to the belly at the front where it will be connected to each other. This ensures the user of the Glans-stripper with the upmost freedom of movement and it prevents him from huge differences in the force by which B is pulling A.

Because the fastener of Design 2 always can be closed fast and faultless while the risk of damaging parts of the body because of the shape of the fastener is brought back to a minimal and because it is easy to let the size be the same every time and because the size is a stable one.

Because the size of Design 3 can be easily adjusted and still be a very stable one. If necessary the size can also be enlarged easily during use and the shape of it reduces the risk of damaging parts of the body.

Because the three Designs can easily be washed with the rest of the dirty laundry and because they take very little space when they are not used.

Because the elastic ribbon with the buttonholes has seperations so small and so strong it will be durable and will not endure the heavy wear and tear common elastic ribbon with buttonholes would do and because part B can be connected to a seperation by wrapping it around one and because the minimal length needed to adjust A with will be limited to just the space needed for K(-) to be able to get through the buttonhole.

Because a large amount of shoe-lace material of 15 meters rolled up around a piece of carton gives the consumer the opportunity to fix the needed length himself in whole and not in part and can do so for several times while the total length of shoe-lace material that has to be purchased by a single consumer will be within an acceptable range for him/her and because it is easier and cheaper than using ready made shoe-laces that most of the times still need to be shortened or even enlarged.

All my versions of K(-) are an improvement because of one or more of the following reasons:

During the act of leading V through K(-) or placing K(-) around V and making K(-) hold on tight to V the handhold K(-) supplies one with will be as much as possible for it's size and if the heads of the screws or the bolts (or the fastener or the hinge) will be countersinked what makes it easier to hold the screwdriver in place it will be as easy and as safe as possible to work with and to make it hold on tight to V. The surface of the insides of part K(-) touching V is bigger what provides K(-) with more grip and it makes K(-) also more suited for a raw or rugged structure on the insides. K(-) can have an extra hole to insert glue. K(-) holds on tight to V with much pressure and makes no use of a mechanism based on a spring or a springlike effect that can not provide the same pressure. K(-) can have two sides that are one because they are connected by a hinge. A clasp could make two sides and a hinge even easier to close and to open. The clasp could be countersinked and easily be opened with for example a tooth of a fork for which the room could be provided for.

Part C, the little archer's bow, does not exist for these designs of the Glans-stripper.

The tubelike parts R to prevent the endings of V from raveling are an improvement because they are made from plastic and they can easily be placed around V and be glued to V by the consumer himself.

A do-it-yourself kit to make the Glans-strippers does not exist and it is an improvement because the consumer can make the Glans-strippers himself easily in the size and manner he wants and he can replace the parts that are worn out and it is cheaper than buying them ready for use.

1. A glans-stripper out of a part A formed by a piece of elastic ribbon whose ends are indissoluble connected to each other and to the middle of part B, the outline of part A being about ⅔ to ¾ of the outline of the root of the penis, the width of the elastic ribbon being about 1 to 1½ cm wherein part B is made out of flat plaited cotton shoe-lace material commonly used in sports shoes, and the ends of part Bare both led through the same spherical shaped and beadlike form K(-) that clenches the shoe-lace_material so tight it will not slip while being used normally but can be moved by a specific action to adjust the size, the length of one half of part B being the outwardly measured distance from the top of the root of the erection to the anus and from the anus to the belly increased width by 25 cm, and the total length of the shoe-lace_material B is twice that distance, part K(-) being configured, in use, to clench B somewhere near the anus.

2.-10. (canceled)
